(19) United States
(12) Patent Application Publication
(10) Pub. No.: US 2025/0288825 A1
(43) Pub. Date: Sep. 18, 2025

(54) Gamma Stimulation Apparatus With Dual Frequencies

(71) Applicant: Aleddra Inc., Renton, WA (US)

(72) Inventors: Chia-Yiu Maa, Bellevue, WA (US); Li-Jyuan Luo, Taipei City (TW)

(21) Appl. No.: 19/227,439

(22) Filed: Jun. 3, 2025

Related U.S. Application Data
(63) Continuation-in-part of application No. 19/186,404, filed on Apr. 22, 2025.
Application No. 19/186,404 is a continuation-in-part of application No. 18/626,148, filed on Apr. 3, 2024.
Application No. 18/626,148 is a continuation-in-part of application No. 18/613,079, filed on Mar. 21, 2024.
Application No. 18/613,079 is a continuation-in-part of application No. 18/408,523, filed on Jan. 9, 2024.

(51) Int. Cl. A61N 5/06

(52) U.S. Cl. CPC A61N 5/0622; A61N 2005/0626; A61N 2005/0653

(57) ABSTRACT

A gamma stimulation apparatus comprises a controller, a first light source, and a second light source. The controller is configured to operate the first light source at a first frequency F1≥60 Hz generating a first light output. Moreover, the controller is configured to operate the second light source at a second frequency F2 (>F1) generating a second light output. When the first light output and the second light output are perceived by a subject simultaneously, an invisible visual simulation at a harmonic frequency (HF) equal to |2F1 −F2| is induced endogenously in a brain of the subject. By carefully choosing F1 and F2, and subsequently HF, the gamma stimulation apparatus may be used for treating or preventing Alzheimer's disease.

The present disclosure is a continuation-in-part (CIP) of U.S. patent application Ser. No. 19/186,404, filed 22 Apr. 2025, which is a CIP of U.S. patent application Ser. No. 18/626,148, filed 3 Apr. 2024, which is a CIP of U.S. patent application Ser. No. 18/613,079, filed 21 Mar. 2024, which is itself is a CIP of U.S. patent application Ser. No. 18/408,523, filed 9 Jan. 2024. Content of aforementioned applications are herein incorporated by reference in their entirety.

### BACKGROUND

### Technical Field

The present disclosure pertains to the field of gamma stimulation apparatus and, more specifically, proposes gamma stimulation apparatus with dual frequencies.

### Description of Related Art

It has been discovered that by flickering a light at a frequency between 35 Hz to 45 Hz or generating a sound at a similar frequency has the effect of stimulating the cells in certain regions of the brain, resulting in using a flicking light or a sound at such a frequency for treating Alzheimer's disease. However, turning on and off a light source at a frequency between 35 Hz to 45 Hz can create visual discomfort in the eyes of a subject. Different approaches have been introduced to overcome this visual discomfort under 40 Hz flickering light.

One of the approaches in U.S. patent application Ser. No. 18/408,523 introduces the use of a controller and two light sources such that the controller would operate these two light sources at two different frequencies resulting a superimposed light operating at a third frequency equal to the difference of these two frequencies. The operating frequency of the first light source is 50 Hz and the operating frequency of the second light source is greater than the operating frequency of the first light source by at least 30 Hz but no more than 65 Hz. In particular, the first frequency is chosen to be 80 Hz and the second frequency 120 Hz, resulting the third frequency to be 40 Hz.

In a research paper entitled “dual-frequency steady-state visual evoked potential for brian computer interface” (https://doi.org/10.1016/j.neulet.2010.07.043), its authors demonstrated that when two groups of LED light sources operating at different frequencies (f1 and f2), a harmonic frequency (HF) equal to 2f1−f2 or 2f2−f1 is induced endogenously in a brain of a subject. In another research paper entitled “investigating of the 2f1−f2 and 2f2−f1 distortion product otoacoustic emissions using a computational model of the gerbil ear” (https://www.sciencedirect.com/science/article/abs/pii/S0378595518300996), its authors confirmed that two different acoustic frequencies (f1 and f2) could also induce an HF equal to 2f1−f2 and 2f2−f1 in a brain of a subject. These research results suggest that the approach of using two frequencies 80 Hz and 120 Hz in U.S. patent application Ser. No. 18/408,523 is just one embodiment of the harmonic frequency induction method based on 2f1−f2 and 2f2−f1 formulas. By carefully choosing f1 and f2 to be flicker-free (60 Hz) and having 2f1−f2 and 2f2−f1 falling in the region of 35 Hz to 45 Hz, it is thus feasible to generalize the frequency requirements introduced in U.S. patent application Ser. No. 18/408,523 (and its CIPs) beyond 80 Hz and 120 Hz while maintaining proper gamma stimulation in the brain of the subject suitable for treating Alzheimer's disease. The present disclosure provides the details of such generalization of gamma stimulation apparatus with dual frequencies.

### SUMMARY

In one aspect, the gamma stimulation apparatus comprises a controller, a first light source, and a second light source. The controller is configured to operate the first light source at a first frequency F1 (≥60 Hz) generating a first light output, and the controller is configured to operate the second light source at a second frequency F2(>F1) generating a second light output. When the first light output and the second light output are perceived by a subject simultaneously, an invisible visual simulation at a harmonic frequency (HF) equal to |2F1−F2| is induced endogenously in a brain of the subject. The first light output is flicker-free for F1≥60 Hz, and the second light output is flicker-free for F2>F1≥60 Hz. Moreover, HF is invisible because it is induced endogenously in the brain of the subject. The gamma stimulation apparatus does not emit such HF. |2F1−F2| refers to the absolute value of the difference of 2F1 and F2. In other words, their difference at 40 Hz and −40 Hz are the same as far as inducing an endogenously gamma stimulation is concerned,

In some embodiments, HF is between 20 Hz and 45 Hz. In practice, HF may be more narrowly chosen to be between 35 Hz and 40 Hz.

In some embodiments, F1 is 80 Hz and F2 is 120 Hz, and thus HF=|2F1−F2|=40 Hz. Similarly, F1 is 80 Hz and F2 is 200 Hz would also generate HF=|2F1−F2|=40 Hz.

In some embodiments, F1 is 65 Hz and F2 is 90 Hz.

In some embodiments, F1 is 70 Hz and F2 is 100 Hz.

In some embodiments, F1 is 75 Hz and F2 is 110 Hz.

In some embodiments, F1 is 85 Hz and F2 is 130 Hz.

In some embodiments, F1 is 90 Hz and F2 is 140 Hz.

Other research has shown that the slow theta stimulation at 4 Hz and 7 Hz has the effect of triggering memory recall. The present disclosure can be easily devised to support slow theta stimulation. In some embodiments, HF is between 3 Hz and 10 Hz.

In some embodiments, F1 is 60 Hz and F2 is 116 Hz, resulting in HF=4 Hz. In some other embodiments, F1 is 60 Hz and F2 is 124 Hz, also resulting in HF=4 Hz.

In some embodiments, F1 is 60 Hz and F2 is 113 Hz, resulting in HF=7 Hz. In some other embodiments, F1 is 60 Hz and F2 is 127 Hz, also resulting in HF=7 Hz.

For the choice of light sources, LED and OLED are preferred because they can operate at a high frequency with accuracy and precision, which is ideal for generating F1, F2, and HF accurately and precisely. Therefore, in some embodiments, the first light source comprises a light emitting diode (LED) or organic LED (OLED), and the second light source comprises another LED or OLED. It is to be noted that the present disclosure is not limited to using only LED or DC-driven LED for the light sources. An AC-driven controller may be designed to drive AC-driven first and second light sources.

In some embodiments, the controller further comprises a circuit to derive F1 and F2 internally, as opposed to receiving F1 and F2 from external sources.

In addition to using LED and OLED as light sources, further measure may be necessary in ensuring the accuracy and precision of HF. This is because inducing an accurate HF (e.g., at 40 Hz) is highly critical in achieving a proper gamma stimulation effect when treating Alzheimer's disease. When HF drifts beyond 40 Hz (e.g., becoming 42 Hz), the effectiveness on treating Alzheimer's disease is greatly reduced or completely vanish. One approach of generating accurate F1 and F2 (and subsequently HF) is using a crystal oscillator circuit in the controller because crystal oscillator is known to generate frequency signal accurately and precisely. Thus, in some embodiments, the circuit comprises a crystal oscillator circuit, and the controller is configured to derive F1 and F2 from the crystal oscillator circuit.

In some embodiments, the circuit comprises a first crystal oscillator circuit and a second crystal oscillator circuit. The controller is configured to derive F1 from the first crystal oscillator circuit and F2 from the second crystal oscillator circuit.

For the gamma stimulation apparatus that consumes, say, less than 20 W, the controller may comprise just a single driver for supplying power to both the first light source and the second light source. However, when the gamma simulation apparatus is over 20 W, it may be more suitable to use two separate drivers for supplying power to the first light source and the second light source separately, thus avoiding the electrical interference between the first light source and the second light source. Therefore, in some embodiments, the controller further comprises a first driver and a second driver. The first driver is configured to operate the first light source at F1 frequency, and the second driver is configured to operate the second light source at F2 frequency.

In another aspect, the gamma stimulation apparatus comprises a rectifier and a control module having a first power output port and a second power output port. The rectifier is configured to convert an external alternating current (AC) power to an internal direct current (DC) power to power the control module. The control module is configured to output, via the first power output port, a first output power having a first periodical waveform at a first frequency F1(≥60 Hz). The control module is also configured to output, via the second power output port, a second output power having a second periodical waveform at a second frequency F2(≥F1) The first power output port is configured to power a first external light source to produce a first light output. The second power output port is configured to power a second external light source to produce a second light output.

When the first light output and the second light output are perceived by a subject simultaneously, an invisible visual simulation at a harmonic frequency (HF) equal to |2F1−F2| is induced endogenously in a brain of the subject. This gamma simulation apparatus may take the form of a two-channel driver for powering two LED light sources in an LED fixture. The terms “first external light source” and “second external light source” may refer to distinct LED, OLED or fixtures positioned at different locations within a system or application. For example, the first and second external light source may comprise separate LED module in the instrument, or light bulbs integrated into a commercial light system.

In some embodiments, HF is between 20 Hz and 45 Hz.

In some embodiments, F1 is 80 Hz and F2 is 120 Hz, and thus HF=|2F1−F2|=40 Hz.

In some embodiments, HF is between 3 Hz and 10 Hz. For example, F1=60 Hz and F2=116 Hz, resulting in HF=4 Hz; or F1=60 Hz and F2=127 Hz, resulting in HF=7 Hz.

In some embodiments, the control module further comprises a circuit to derive F1 and F2 internally, as opposed to receiving F1 and F2 from external sources.

In some embodiments the circuit comprises a crystal oscillator circuit, and the control module is configured to derive F1 and F2 from the crystal oscillator circuit.

In some embodiments, the circuit comprises a first crystal oscillator circuit and a second crystal oscillator circuit. The control module is configured to obtain F1 from the first crystal oscillator circuit and F2 from the second crystal oscillator circuit

### BRIEF DESCRIPTION OF THE DRAWINGS

The accompanying drawings are included to aid further understanding of the present disclosure and are incorporated in and constitute a part of the present disclosure.

The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts an embodiment of the present disclosure using one rectifier, one driver, and two light sources.

FIG. 2 schematically depicts an embodiment of the present disclosure using two rectifiers, two drivers, and two light sources.

FIG. 3 schematically depicts an embodiment of the present disclosure using one rectifier and one control module.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of gamma stimulation apparatuses having different form factors.

A gamma stimulation apparatus comprises a controller, a first light source, and a second light source. The controller is configured to operate the first light source at a first frequency F1≥60 Hz generating a first light output. Moreover, the controller is configured to operate the second light source at a second frequency F2(>F1) generating a second light output. When the first light output and the second light output are perceived by a subject simultaneously, an invisible visual simulation at a harmonic frequency (HF) equal to |2F1−F2| is induced endogenously in a brain of the subject.

### Example Implementations

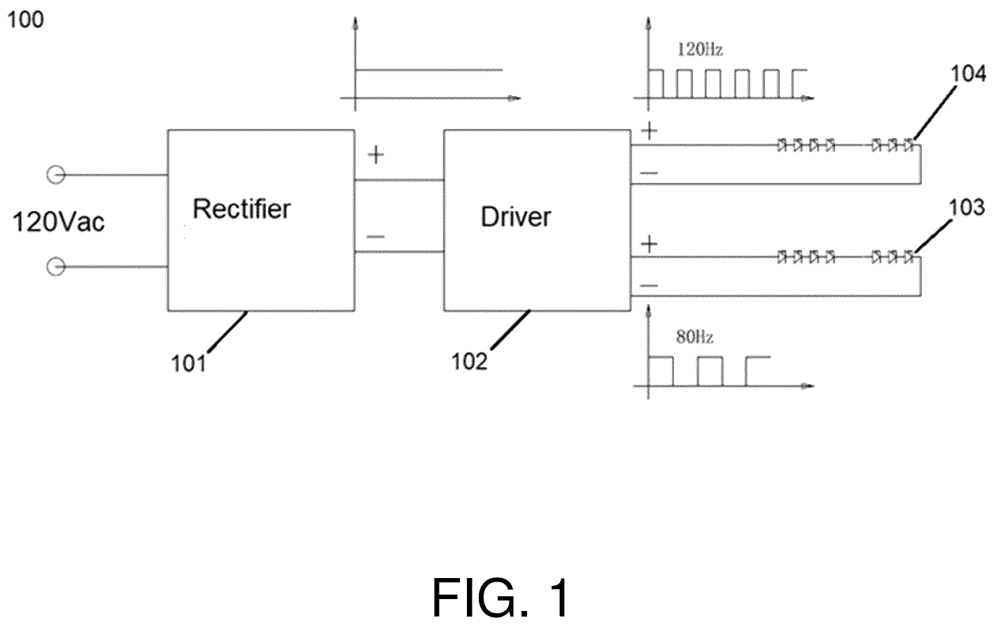

FIG. 1 shows an embodiment of the gamma stimulation apparatus of the present disclosure 100. It comprises a rectifier 101, a driver 102, a first light source 103, and a second light source 104. The rectifier 101 and the driver 102 together form the controller.

The rectifier 101 converts an external AC power to an internal DC power to power driver 102. The driver 102, which is part of the controller, operates the first light source 103 at F1=80 Hz to generate a first light output and the second light source 104 at F2=120 Hz to generate the second light output. When the first light output and the second light output are perceived by a subject simultaneously, an invisible visual simulation at a harmonic frequency (HF) equal to |2F1−F2|=2*80 Hz−120 Hz=40 Hz is induced endogenously in a brain of the subject. The first light output is flicker-free for F1=60 Hz, and the second light output is flicker-free for F2=120 Hz. Moreover, HF is invisible because it is induced endogenously in the brain of the subject. The gamma stimulation apparatus does not emit such HF.

Other combinations may be chosen for F1 and F2, such as (65 Hz, 90 Hz), (70 Hz, 100 Hz), (75 Hz, 110 Hz), (85 Hz, 130 Hz), (90 Hz, 140 Hz), etc.

The embodiment 100 can be revised to support slow theta stimulation by setting F1=60 Hz and F2=116 Hz (or F2=124 Hz), resulting in HF=4 Hz; or F1=60 Hz and F2=113 Hz (or F2=127 Hz), resulting in HF=7 Hz.

The first light source 103 and the second light source are DC-driven LED. The present disclosure is not limited to using only LED or DC-drive LED for light sources. An AC-driven controller may be designed to drive AC-driven first and second light sources, without using any rectifier.

Though not shown in FIG. 1, the driver 102 may comprise a crystal oscillator circuit, which is used to derive F1 and F2 internally. Alternatively, the driver 102 may comprise a first crystal oscillator circuit and a second crystal oscillator circuit, such that the driver 102 uses the first crystal oscillator circuit to derive F1 frequency for driving the first light source 103 and the second crystal oscillator circuit to derive F2 frequency for driving the second light source 104.

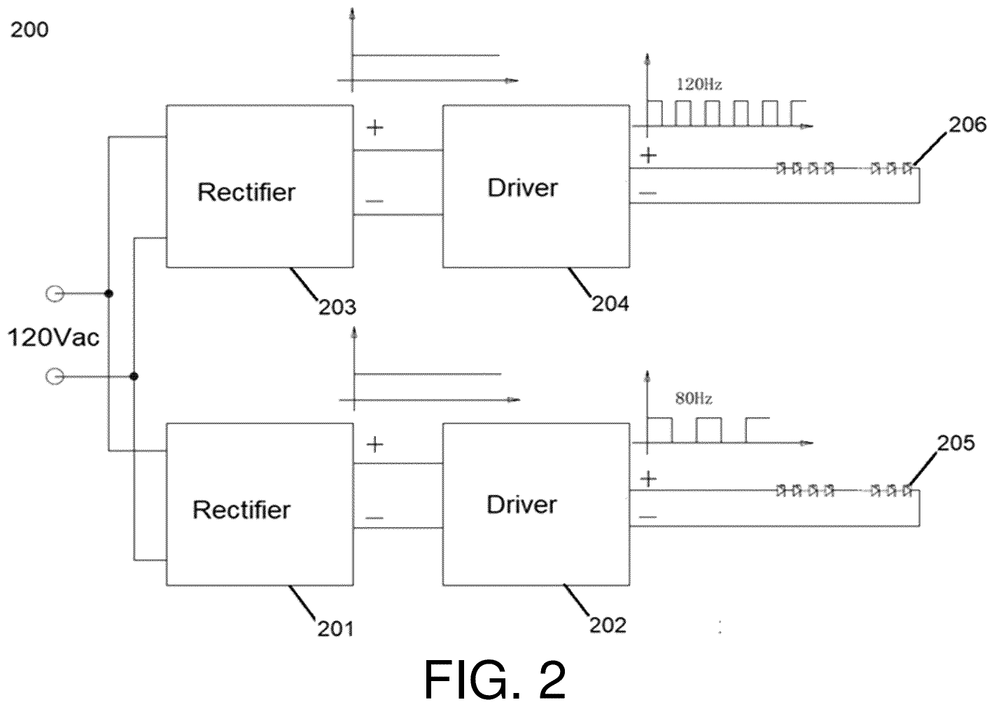

FIG. 2 shows another embodiment of the gamma stimulation apparatus of the present disclosure 200. It comprises a first rectifier 201, a first driver 202, a second rectifier 203, a second driver 204, a first light source 205, and a second light source 206. The first rectifier 201, the first driver 202, the second rectifier 203, and the second driver 204 together form the controller. The first rectifier 201 converts an external AC power to an internal DC power to power the first driver 202. Similarly, the second rectifier 203 converts an external AC power to an internal DC power to power the second driver 204.

The first driver 202 operates the first light source 205 at F1=80 Hz to generate a first light output, and the second driver 204 operates the second light source 206 at F2=120 Hz to generate the second light output. When the first light output and the second light output are perceived by a subject simultaneously, an invisible visual simulation at HF equal to |2F1−F2|=2*80 Hz−120 Hz=40 Hz is induced endogenously in a brain of the subject.

Other combinations may be chosen for F1 and F2, such as (65 Hz, 90 Hz), (70 Hz, 100 Hz), (75 Hz, 110 Hz), (85 Hz, 130 Hz), (90 Hz, 140 Hz), etc.

The embodiment 200 can be revised to support slow theta stimulation by setting F1=60 Hz and F2=116 Hz (or F2=124 Hz), resulting in HF=4 Hz; or F1=60 Hz and F2=113 Hz (or F2=127 Hz), resulting in HF=7 Hz.

Though not shown in FIG. 2, the driver 202 may comprise a crystal oscillator circuit which is used to derive F1 frequency. Similarly, the driver 204 may comprise another oscillator circuit which is used to derive F2 frequency.

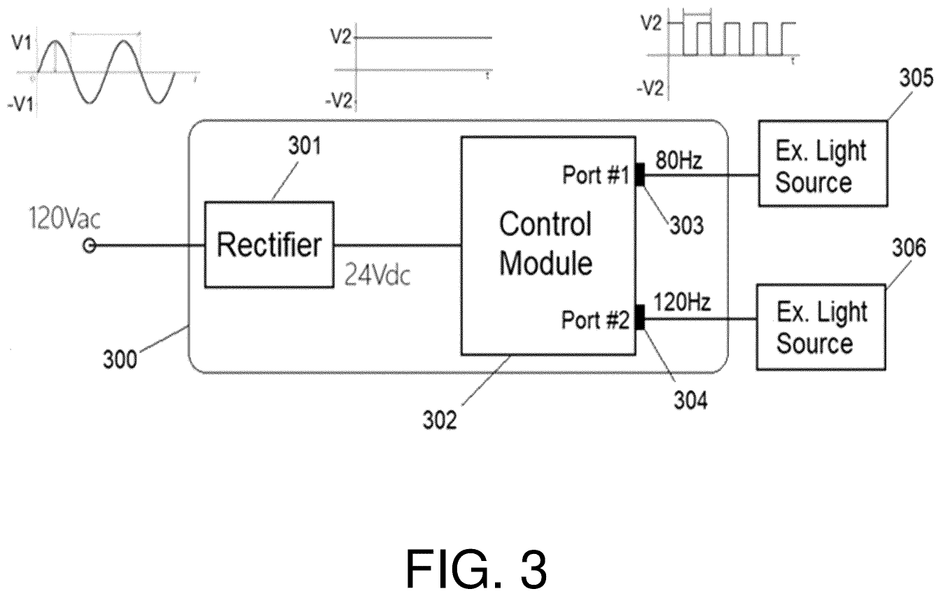

FIG. 3 shows another embodiment of the gamma stimulation apparatus of the present disclosure 300. It comprises a rectifier 301 and a control module 302 having a first power output port 303 and a second power output port 304. The rectifier 301 converts an external AC power to an internal DC power to power the control module 302. The control module 302 outputs via the first power output port 303 a first output power having a first periodical waveform (e.g., a square waveform) at a first frequency F1=80 Hz. The control module 302 outputs via the second power output port 304 a second output power having a second periodical waveform (e.g., a square waveform) at a second frequency F2=120 Hz. The first power output port 303 connects and powers a first external light source 305 to produce a first light output, and the second power output port 304 connects and powers a second external light source 306 to produce a second light output. When the first light output and the second light output are perceived by a subject simultaneously, an invisible visual simulation at HF equal to |2F1−F2|=2*80 Hz−120 Hz=40 Hz is induced endogenously in a brain of the subject. The first light output is flicker-free for F1=80 Hz, and the second light output is flicker-free for F2=120 Hz. Moreover, HF is invisible because it is induced endogenously in the brain of the subject. The gamma stimulation apparatus does not emit such HF.

The embodiment 300 can be revised to support slow theta stimulation by setting F1=60 Hz and F2=116 Hz (or F2=124 Hz), resulting in HF=4 Hz; or F1=60 Hz and F2=113 Hz (or F2=127 Hz), resulting in HF=7 Hz.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A gamma stimulation apparatus, comprising:
a controller;
a first light source; and
a second light source,
wherein:
the controller is configured to operate the first light source at a first frequency (F1)≥60 Hz to generate a first light output,
the controller is configured to operate the second light source at a second frequency (F2)>F1 to generate a second light output, and
when the first light output and the second light output are perceived by a subject simultaneously, an invisible visual simulation at a harmonic frequency (HF) equal to |2F1−F2| is induced endogenously in a brain of the subject.

2. The apparatus of claim 1, wherein HF is between 20 Hz and 45 Hz.

3. The apparatus of claim 2, wherein F1 is 80 Hz and F2 is 120 Hz.

4. The apparatus of claim 2, wherein F1 is 65 Hz and F2 is 90 Hz.

5. The apparatus of claim 2, wherein F1 is 70 Hz and F2 is 100 Hz.

6. The apparatus of claim 2, wherein F1 is 75 Hz and F2 is 110 Hz.

7. The apparatus of claim 2, wherein F1 is 85 Hz and F2 is 130 Hz.

8. The apparatus of claim 2, wherein F1 is 90 Hz and F2 is 140 Hz.

9. The apparatus of claim 1, wherein HF is between 3 Hz and 10 Hz.

10. The apparatus of claim 9, wherein F1 is 60 Hz and F2 is 116 Hz.

11. The apparatus of claim 9, wherein F1 is 60 Hz and F2 is 124 Hz.

12. The apparatus of claim 9, wherein F1 is 60 Hz and F2 is 113 Hz.

13. The apparatus of claim 9, wherein F1 is 60 Hz and F2 is 127 Hz.

14. The apparatus of claim 1, wherein the first light source comprises a light emitting diode (LED) or organic LED (OLED), and wherein the second light source comprises another LED or OLED.

15. The apparatus of claim 1, wherein the controller further comprises a circuit configured to derive F1 and F2 internally.

16. The apparatus of claim 10, wherein the circuit comprises a crystal oscillator circuit, wherein the controller is configured to obtain F1 and F2 from the crystal oscillator circuit.

17. The apparatus of claim 10, wherein the circuit comprises a first crystal oscillator circuit and a second crystal oscillator circuit, and wherein the controller is configured to obtain F1 from the first crystal oscillator circuit and F2 from the second crystal oscillator circuit.

18. The apparatus of claim 1, wherein the controller further comprises a first driver and a second driver, wherein the first driver is configured to operate the first light source at F1, and wherein the second driver is configured to operate the second light source at F2.

19. A gamma stimulation apparatus, comprising:
a rectifier; and
a control module having a first power output port and a second power output port,
wherein:
the rectifier is configured to convert an external alternating current (AC) power to an internal direct current (DC) power to power the control module,
the control module is configured to output, via the first power output port, a first output power having a first periodical waveform at a first frequency (F1)≥60 Hz,
the control module is configured to output, via the second power output port, a second output power having a second periodical waveform at a second frequency (F2)>F1,
the first power output port is configured to power a first external light source to produce a first light output,
the second power output port is configured to power a second external light source to produce a second light output, and
when the first light output and the second light output are perceived by a subject simultaneously, an invisible visual simulation at a harmonic frequency (HF) equal to |2F1−F2| is induced endogenously in a brain of the subject.

20. The apparatus of claim 19, wherein HF is between 20 Hz and 45 Hz.

21. The apparatus of claim 20, wherein F1 is 80 Hz and F2 is 120 Hz.

22. The apparatus of claim 19, wherein HF is between 3 Hz and 10 Hz.

23. The apparatus of claim 19, wherein the control module further comprises a circuit to derive F1 and F2 internally.

24. The apparatus of claim 23, wherein the circuit comprises a crystal oscillator circuit, and wherein the control module is configured to obtain F1 and F2 from the crystal oscillator circuit.

25. The apparatus of claim 23, wherein the circuit comprises a first crystal oscillator circuit and a second crystal oscillator circuit, and wherein the control module is configured to obtain F1 from the first crystal oscillator circuit and F2 from the second crystal oscillator circuit.
